(19) United States
(12) Patent Application Publication
(10) Pub. No.: US 2026/0001826 A1
(43) Pub. Date: Jan. 1, 2026

(54) METHOD FOR PRODUCING AN ALKALI METAL ALKOXIDE

(71) Applicant: Evonik Operations GmbH, Essen (DE)

(72) Inventors: Marius Mewald, Mettmann (DE); Sebastian Fuß, Flieden (DE); Marco Wolfstädter, Woerth am Main (DE); Karim Hamidie Ashtiani, Hanau (DE)

(73) Assignee: Evonik Operations GmbH, Essen (DE)

(21) Appl. No.: 19/120,523

(22) PCT Filed: Oct. 12, 2023
(86) PCT No.: PCT/EP2023/078275

§ 371 Date: Apr. 11, 2025

(30) Foreign Application Priority Data
Oct. 19, 2022 (EP) 22202368.1

(51) Int. Cl. C07C 29/70; C07C 31/30

(52) U.S. Cl. CPC C07C 29/70; C07C 31/30

(57) ABSTRACT

A method of producing an alkali metal alkoxide involves the following steps: (i) providing an organic compound having at least one-OH group and having a molecular weight of 75 to 750 g/mol in a reaction vessel in an inert atmosphere; (ii) adding a C₁₋₆ alkali metal alcoholate to obtain a reaction mixture; (iii) heating the reaction mixture of step (ii) under stirring to 25 to 180° C. to obtain the alkali metal alkoxide and a C₁₋₆ alcohol; and (iv) distilling off the obtained C₁₋₆ alcohol. While distilling, at least one non-polar solvent having a boiling point higher than the obtained alcohol is continuously or batchwise added to the mixture.

The present invention refers to a method of producing an alkali metal alkoxide comprising the following steps:
i) providing an organic compound having at least one —OH group and having a molecular weight of 75 to 750 g/mol in a reaction vessel in an inert atmosphere;
ii) adding a C₁₋₆ alkali metal alcoholate to obtain a reaction mixture;
iii) heating the reaction mixture of step ii) under stirring to 25 to 180° C. to obtain the alkali metal alkoxide and a C₁₋₆ alcohol; and
iv) distilling off the obtained C₁₋₆ alcohol; wherein while distilling at least one non-polar solvent having a boiling point higher than the obtained alcohol is continuously or batchwise added to the mixture.

Alkali metal alkoxide are used for a variety of purposes, such as aldol additions, esterifications/transesterifications, malonic ester syntheses, ether formations and other general use as a base. The alkali metal alkoxides are also widely used in the food industry such as in margarine production or vitamin A synthesis, in pharmaceutical chemistry such as in the production of antibiotics, pain relievers, chemotherapeutics and epileptics, in agrochemistry such as in the production of herbicides and fungicides and in many other areas of application such as in the production of optical brighteners, UV absorbers and photoinitiators.

Different processes are known in the state of the art to produce alkali metal alkoxide.

One known process comprises the reaction from an alcohol and an alkali metal. During this reaction hydrogen is produced, wherein the release of hydrogen can increase rapidly with low-molecular alcohols (Rompp Online. Georg Thieme Verlag, retrieved on 28. Mai 2020).

Another alternative comprises the reaction of an alcohol with a strong base, such as alkali amides or alkali hydrides. With this reaction, ammonia or hydrogen are produced as by-products. Since the alkali amides are very sensitive, the product yield is strongly fluctuating.

Another alternative process as described in EP 1195369 A1 comprises the reaction of an alcohol with alkali amalgam. After the reaction the final product is contaminated with hydrogen and mercury. Further development of this process as disclosed in EP 0776995 A1 comprises reaction of the corresponding alcohol with another alcoholate in the liquid phase in the presence of an electric field. Metal ions are introduced under the influence of the electric field so as to give the required alcoholate, preferably with the decomposition of the other alcoholate and the subsequent introduction of the metal into the alcohol being affected in different zones separated by an ion-exchange membrane.

Another alternative process, which is described in U.S. Pat. No. 3,418,383 discloses the production of alkali metal alcoholates by an improved interchange reaction between a lower alcoholate of an alkali metal and a higher alcohol to produce a higher alcoholate of the alkali metal, wherein the interchange reaction is performed in the presence of vapors of higher alcohols passing through the interchange reaction mass. The reaction time is very high for this reaction and a high molar amount of alcohol is needed for this reaction.

However, all the known processes lead to alkali metal alkoxides containing impurities, which can lead to a lower storage stability of the obtained alkali metal alkoxide and consequently require a labour-intensive purification process or are not desirable to be used as starting material for pharmaceutical applications.

Therefore, it was an objective of the present invention to provide an improved method yielding in alkali metal oxides having low impurities and which does not require a separate additional purification process.

This is achieved by the method according to the present invention, which achieves a complete conversion of the starting compounds including the C₁₋₆ alkali metal alcoholate and thus a high purity of the resulting alkali metal alkoxide, without the need of a complex separation as in methods known in the art.

In particular, the object is achieved by a method of producing an alkali metal alkoxide comprising the following steps:
i) providing an organic compound having at least one-OH group and having a molecular weight of 75 to 750 g/mol in a reaction vessel in an inert atmosphere;
ii) adding a C₁₋₆ alkali metal alcoholate to obtain a reaction mixture;
iii) heating the reaction mixture of step ii) under stirring to 25 to 180° C. to obtain the alkali metal alkoxide and a C₁₋₆ alcohol; and
iv) distilling off the obtained C₁₋₆ alcohol; wherein while distilling at least one non-polar solvent having a boiling point higher than the obtained alcohol is continuously or batchwise added to the mixture.

In order to achieve a complete conversion, it is required that while distilling at least one non-polar solvent having a boiling point higher than the obtained alcohol is continuously or batchwise added to the mixture. The resulting alkali metal alkoxide has a high purity and is isolated as a liquid without the need of further purification or as a crystalline powder with good flow properties, which was easy to filtrate.

The distillation can be any common distillation method known in the art, preferably it is an azeotropic, reactive, vacuum, or fractional distillation or a combination of minimum two of the beforementioned techniques.

While performing the distillation an inert gas stream, preferably a nitrogen stream, can be applied, preferably with a flow rate of 10 to 300 NL/h, more preferably 50 to 200 NL/h.

“At least one” means one or more, i.e. 1, 2, 3, 4, 5, 6, 7, 8, 9 or more. “At least one”, as used herein in relation to any component, refers to the number of chemically different molecules, i.e. to the number of different types of the referenced species, but not to the total number of molecules. For example, “at least one non-polar solvent” means that at least one type of molecule falling within the definition for a non-polar solvent is used but that also two or more different types of non-polar solvents falling within this definition can be present but does not mean that only one or more molecules of one type of non-polar solvent are present.

In particular, the present invention refers to:
1. A method of producing an alkali metal alkoxide, more preferably a potassium alkoxide, comprising or consisting of the following steps:
   i) providing an organic compound having at least one-OH group and having a molecular weight of 75 to 750 g/mol, preferably 75 to 350 g/mol, in a reaction vessel in an inert atmosphere;
   ii) adding a C₁₋₆ alkali metal alcoholate, more preferably a C₁₋₄ alkali metal alcoholate, more preferably a C₁₋₄ potassium alcoholate, to obtain a reaction mixture;
   iii) heating the reaction mixture of step ii) under stirring to 25 to 180° C., preferably to 60 to 170° C. to obtain the alkali metal alkoxide and a C₁₋₆ alcohol, preferably a C₁₋₄ alcohol; and
   iv) distilling off the obtained C₁₋₆ alcohol, preferably C₁₋₄ alcohol; wherein while distilling at least one non-polar solvent having a boiling point higher than the obtained alcohol, preferably of at least 138°, more preferably 138° C. to 300° C., is continuously or batchwise added to the mixture, preferably in up to four batches.

If step iii) is performed at temperatures of 170° C. or lower the purity of the resulting alkali metal alkoxide is further increased.
2. The method of item 1, wherein the organic compound having at least one-OH group is selected from substituted or unsubstituted aliphatic or aromatic hydrocarbons, silyl ethers, polyols, ethers, and polyethers or mixtures thereof, preferably, the organic compound is selected from phenols, benzylic alcohols, 2-alkoxyethanols, 2-aryloxyethanols, 2-siloxyethanols, 2-(2-alkoxyethoxy) ethanols, 2-(2-aryloxyethoxy) ethanol, 2-(2-silanyloxyethoxy) ethanols, 3-alkoxypropanols, 3-aryloxypropanols, 3-siloxypropanols, 2-(2-alkoxyethoxy) ethanols, 2-(2-aryloxyethoxy) ethanol, 2-(2-silanyloxyethoxy) ethanols, diols, triols, tetraols, more preferably, the compound is selected from 2-benzyloxyethanol, 2-methoxyethanol, diethylenglycolmonobenzylether, 2-tert-butoxyethanol, 2-(2-methoxyethoxy) ethanol, 2-(tert-butyl-dimethylsilanyloxy) ethanol, 2-(tert-butyldiphenylsilanyloxy) ethanol, 2-(2-(tert-butyldimethylsilylethoxy) ethanol, 2-(2-tert-butyldiphenylsilylethoxy) ethanol, pentaerythritol, triethylenglycol, tetraethylenglycol, 2,2′-((2-((2-hydroxyethoxy) methyl)-2-(hydroxymethyl) propane-1,3-diyl) bis (oxy)) bis (ethan-1-ol), 3-methoxypropanol or mixtures thereof. Preferably, the organic compound having at least one —OH group is selected from phenols, benzylic alcohols, 2-alkoxyethanols, 2-aryloxyethanols, 2-siloxyethanols, 2-(2-alkoxyethoxy) ethanols, 2-(2-aryloxyethoxy) ethanol, 2-(2-silanyloxyethoxy) ethanols, 3-alkoxypropanols, 3-aryloxypropanols, 3-siloxypropanols, 2-(2-alkoxyethoxy) ethanols, 2-(2-aryloxyethoxy) ethanol, 2-(2-silanyloxyethoxy) ethanols, diols, triols, tetraols. More preferably, the organic compound having at least one —OH group is selected from 2-benzyloxyethanol, 2-methoxyethanol, diethylenglycolmonobenzylether, 2-tert-butoxyethanol, 2-(2-methoxyethoxy) ethanol, 2-(tert-butyl-dimethylsilanyloxy) ethanol, 2-(tert-butyldiphenylsilanyloxy) ethanol, 2-(2-(tert-butyldimethylsilylethoxy) ethanol, 2-(2-tert-butyldiphenylsilylethoxy) ethanol, pentaerythritol, triethylenglycol, tetraethylenglycol, 2,2′-((2-((2-hydroxyethoxy) methyl)-2-(hydroxymethyl) propane-1,3-diyl) bis (oxy)) bis (ethan-1-ol), 3-methoxypropanol.
3. The method of any of the preceding items, wherein the C₁₋₆ alkali metal alcoholate is selected from potassium methanolate, potassium ethanolate, potassium 2-methylpropan-2-olate, potassium propan-2-olate, potassium propan-1-olate, potassium butan-2-olate, and potassium tert-butoxide.
4. The method of any of the preceding items, wherein the at least one non-polar solvent is selected from toluene, dodecane, tetradecane, xylenes, or tetraethylene glycol dimethyl ether or mixtures thereof. Preferably the at least one solvent is selected from toluene, xylenes, cumene, biphenyl, octane, nonane, C₁₀₋₂₀ alkanes and isomeric forms thereof, C₁₀₋₂₀ decanes and isomeric forms thereof, ethylene glycol dimethyl ether, diethylene glycol dimethyl ether, triethylene glycol dimethyl ether, tetraethylene glycol dimethyl ether, ethylene glycol diethyl ether, diethylene glycol diethyl ether, triethylene glycol diethyl ether, tetraethylene glycol diethyl ether or mixtures thereof.
5. The method of any of the preceding items, wherein the reactor comprises a rotating element for the stirring; preferably

The at least one non-polar solvent is added, when the moment of force M of the rotating element has increased by a factor of at least 1.5 and the moment of force M is reduced after addition of the at least one non-polar solvent by a factor of at least 1.5.
6. Method according to any one of the preceding items, wherein the at least one non-polar solvent is present in an amount of 1 ml/mmol to 20 ml/mmol, preferably between 1 ml/mmol and 10 ml/mmol of the organic compound having at least one-OH group having a molecular weight of 75 to 750 g/mol, preferably 75 to 350 g/mol.
7. Method according to any one of the preceding items, wherein the reaction time is at least 3 hours, preferably at least 24 hours for the batchwise addition or at least 0.5 h, preferably 6 h for the continuous addition.
8. Method according to any one of the preceding items, wherein the at least one non-polar solvent is washed out after the reaction with a volatile non-polar solvent, preferably the volatile non-polar solvent is selected from methyl tert-butyl ether and n-pentane.
9. Method according to any one of the preceding items, wherein the organic compound having at least one —OH group having a molecular weight of 75 to 750 g/mol, preferably 75 to 350 g/mol, and the C₁₋₆ alkali metal alcoholate, C₁₋₄ alkali metal alcoholate, are present in a molar ratio of 2:1 to 1:1.

In one embodiment of the present invention, the reaction vessel comprises a rotating element and
the at least one non-polar solvent is added, when the moment of force M of the rotating element has increased by a factor of at least 1.5 and
the moment of force M is reduced after addition of the at least one non-polar solvent by a factor of at least 1.5.

In one embodiment of the present invention, the at least one non-polar solvent is present in an amount of 1 ml/mmol to 20 ml/mmol, preferably between 1 ml/mmol and 10 ml/mmol. In another embodiment the at least one solvent is non-polar and aprotic, preferably selected from aromatic hydrocarbons, aliphatic hydrocarbons, ether solvents or mixtures thereof.

In a preferred embodiment, the at least one non-polar solvent is a hydrocarbon with at least 4 carbon atoms, preferably selected from toluene, dodecane, tetradecane, xylenes, tetraethylene glycol dimethyl ether, or a silane or siloxane.

In another preferred embodiment, the reaction time is at least 12 hours, preferably at least 24 hours. In one preferred embodiment step iv) is performed for 4 to 5 hours.

In another embodiment the reaction after the reaction time is cooled with a cooling ramp of approximately 1 K/min to room temperature.

In another preferred configuration, the at least one non-polar solvent is washed out after the reaction with a volatile non-polar solvent. The volatile non-polar solvent is preferably an aprotic solvent, more preferably selected from MTBE, toluene, pentane, or hexane.

In another embodiment, the resulting alkali metal alkoxide is dried under inert atmosphere and under the application of vacuum at room temperature for not less than 5 h.

The obtained alkali metal alkoxides can have an assay of at least 90%, preferably at least 95%, more preferably at least 99%, as determined by titration.

### EXAMPLES

The production of alkali metal alkoxides by reaction of organic compound having at least one —OH group and C₁₋₆ alkali metal alcoholates.

### General Method

The reaction equipment is inertized with inert gas and kept water-free prior conducting the reaction. The organic compound having at least one —OH group (1 Eq.) is charged into the reactor, at least one C₁₋₆ alkali metal alcoholate is added, for example methanolate (1 Eq.), handling under inert atmosphere is essential. For the reaction start the flask is heated introducing inert gas into the mass. The gas flow is kept for the whole duration of reaction.

Afterwards, the obtained C₁₋₆ alcohol is distilled off for complete conversion (IPC) IPC: NMR for C₁₋₆ alcohol.

During the distillation stirring is getting difficult, as viscosity is getting very high (indicated by increase of stirrer torque). The viscosity is reduced by addition of at least one non-polar solvent continuously or batch wise. After the distilling step a slurry containing the alkali metal alkoxide is obtained.

Cooling ramp (for laboratory trials) is maintained at −0.1 to 1 K/min to a minimum of room temperature. The cooling procedure must be adapted for considered equipment and the skilled person is well able to perform this within his general knowledge.

For washing the slurry is charged with pressure onto a filter and the filter cake is washed two times with non-polar solvent. The final product is dried with inert gas under vacuum.

### Reaction of Benzyloxy Ethanol to Potassium-Benzyloxy Ethanolate in Presence of N-Dodecane

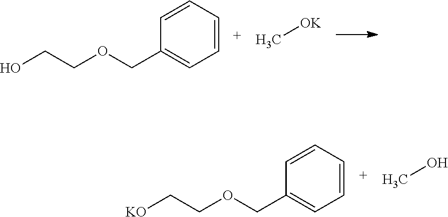

The desired reaction equipment is inertized with inert gas and kept water-free prior conducting the reaction. 627.7 g benzyloxyethanol (99%; 4 mol; 1 Eq.) were charged into reactor, and 166.8 g potassium methanolate (98.95; 4 mol; 1 Eq.) where added, under inert (dry atmosphere). (Re)-inertization of reactor after addition of raw materials. The reaction was heated to 110° C. with constant stirring and nitrogen was started to be introduced into the mass. A 3-blade propeller stirring shaft with a total blade diameter of 75 mm was used. The nitrogen flow is maintained to approx. 100 L/h dry nitrogen for the whole duration of distillation. The methanol was distilled off for approx. 160 h reaction time.

Full conversion is checked via NMR, with residual methanol content.

During distillation, stirring (rpm 100) becomes difficult because the viscosity becomes very high. The torque increases from 0-4 Ncm to 15-30 Ncm. The viscosity can be reduced by adding n-dodecane, (0.1-0.2 L) for a homogeneous reaction mixture, the torque decreases by the addition to 2-6 Ncm. n-dodecane is carried out at the reactor head as an aerosol during the reaction time and must be replenished in several portions. combined amount of n-dodecane (100%; 0.25 L; 1.1 mol).

At the end of distillation, 250 ml (anhydrous/100%) toluene were added for preparation of a slurry (at approx. 100° C. and full speed agitation). Toluene has to be dried to a low water content before addition (e.g., with molsieve, AlOx or another appropriate method), or purchased in anhydrous form.

Cooling ramp (for laboratory trials) is maintained at −0.1 to 1 K/min to a minimum of 20° C. The cooling procedure must be adapted for considered equipment.

For washing the slurry is charged with pressure onto a filter. Residuals are transferred with 200 ml toluene (anhydrous) onto cake and filter cake is washed two times with 125 mL toluene (anhydrous), each. Afterwards, the filter cake was washed twice with 200 mL dry MTBE, each. The final product is dried with inert gas application and vacuum at RT. Yield: 91.3% Purity Titration: 98.9 wt.-%.

### Reaction of Benzyloxyethanol to Potassium-Benzyloxyethanolate in Presence of N-Tetradecane

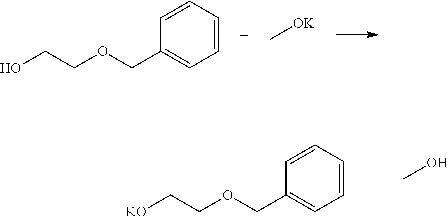

The desired reaction equipment is inertized and kept water-free prior conducting the reaction. 310.6 g benzyloxyethanol (99%; 2 mol; 1 Eq.) were charged into reactor, and 144.6 g potassium methanolate (98.95%; 2 mol; 1 Eq.) where added, under inert (dry atmosphere). (Re)-inertization of reactor after addition of raw materials. The reaction was heated to 120° C. with constant stirring and nitrogen was started to be introduced into the mass. A 3-blade propeller stirring shaft with a total blade diameter of 75 mm was used. The nitrogen flow is maintained to approx. 100 L/h dry nitrogen for the whole duration of distillation. The methanol was distilled off for approx. 168 h reaction time.

Full conversion is checked via NMR, with residual methanol content.

During distillation, stirring (rpm 100) becomes difficult because the viscosity becomes very high. The torque increases from 0-5 Ncm to 10-18 Ncm. The viscosity can be reduced by adding n-tetradecane, (0.1-0.2 L) for a homogeneous reaction mixture, the torque decreases by the addition to 2-4 Ncm. n-tetradecane is carried out at the reactor head as an aerosol during the reaction time and must be replenished in several portions. Combined amount of n-tetradecane (100%; 0.45 L; 1.7 mol)

At the end of distillation, 50 mL (anhydrous/100%) toluene were added for preparation of a slurry (at approx. 100° C. and full speed agitation). Toluene has to be dried to a low water content before addition (e.g., with molsieve, AlOx or another appropriate method), or purchased in anhydrous form.

Cooling ramp (for laboratory trials) is maintained at −0.1 to 1 K/min to a minimum of 20° C. The cooling procedure must be adapted for considered equipment.

For washing the slurry is charged with pressure onto a filter. Residuals are transferred with 25 ml toluene (anhydrous) onto cake and filter cake is washed three times with 25 mL toluene (anhydrous), each. Afterwards, the filter cake was washed twice with 200 mL dry MTBE, each. The final product is dried with inert gas application and vacuum at RT. Yield: 92.4% Purity Titration: 98.5 wt.-%.

Reaction of Benzyloxyethanol to Potassium-Benzyloxyethanolate in Presence of o-Xylene

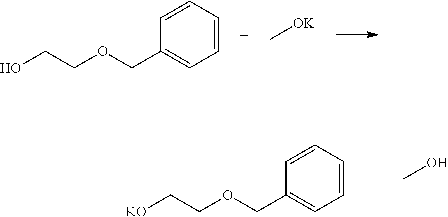

The desired reaction equipment is inertized and kept water-free prior conducting the reaction. 415.6 g benzyloxyethanol (99%; 2,7 mol; 1 Eq.) were charged into reactor, and 193.4 g potassium methanolate (98.95%; 2.7 mol; 1 Eq.) where added, under inert (dry atmosphere). (Re)-inertization of reactor after addition of raw materials. The reaction was heated to 120° C. with constant stirring and nitrogen was started to be introduced into the mass. A 3-blade propeller stirring shaft with a total blade diameter of 75 mm was used. The nitrogen flow is maintained to approx. 100 L/h dry nitrogen for the whole duration of distillation. The methanol was distilled off for approx. 360 h reaction time.

Full conversion is checked via NMR, with residual methanol content.

During distillation, stirring (rpm 100) becomes difficult because the viscosity becomes very high.

The torque increases from 0-5 Ncm to 10-22 Ncm. The viscosity can be reduced by adding o-xylene, (0.1-0.4 L) for a homogeneous reaction mixture, the torque decreases by the addition to 5-7 Ncm. o-xylene is carried out at the reactor head as an aerosol during the reaction time and must be replenished in several portions. combined amount of o-xylene (100%; 1.0 L; 8.3 mol).

At the end of distillation, 50 mL (anhydrous/100%) toluene were added for preparation of a slurry (at approx. 100° C. and full speed agitation). Toluene has to be dried to a low water content before addition (e.g., with molsieve, AlOx or another appropriate method), or purchased in anhydrous form.

Cooling ramp (for laboratory trials) is maintained at −0.1 to 1 K/min to a minimum of 20° C. The cooling procedure must be adapted for considered equipment.

For washing the slurry is charged with pressure onto a filter. Residuals are transferred with 25 ml toluene (anhydrous) onto cake and filter cake is washed three times with 25 mL toluene (anhydrous), each. Afterwards, the filter cake was washed twice with 100 mL (dry, water content <10ppm) MTBE, each. The final product is dried with inert gas under application and vacuum (RT up to 35° C., p=0.05 bara, t=20 h). Yield: 92.8% Purity Titration: 98.7 wt.-%.

### Reaction of Benzyloxyethanol to Potassium-Benzyloxyethanolate in Presence of Tetraglyme

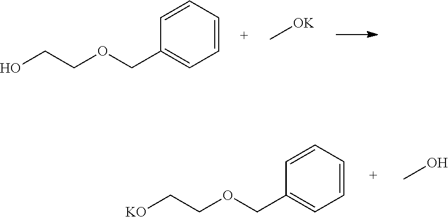

The desired reaction equipment is inertized and kept water-free prior conducting the reaction. 311.2 g benzyloxyethanol (99%; 2.0 mol; 1 Eq.) were charged into reactor, and 143.4 g potassium methanolate (98.95%; 2.0 mol; 1 Eq.) where added, under inert (dry atmosphere). (Re)-inertization of reactor after addition of raw materials. The reaction was heated to 120° C. with constant stirring and nitrogen was started to be introduced into the mass. A 3-blade propeller stirring shaft with a total blade diameter of 75 mm was used. The nitrogen flow is maintained to approx. 100 L/h dry nitrogen for the whole duration of distillation. The methanol was distilled off for approx. 264 h reaction time.

Full conversion is checked via NMR, with residual methanol content.

During distillation, stirring (rpm 100) becomes difficult because the viscosity becomes very high. The torque increases from 0-6 Ncm to 10-26 Ncm. The viscosity can be reduced by adding tetraglyme, (0.1 L) for a homogeneous reaction mixture, the torque decreases by the addition to 7-14 Ncm. Tetraglyme is carried out at the reactor head as an aerosol during the reaction time and must be replenished in several portions. combined amount of tetraglyme (100%; 0.6 L; 2.1 mol).

At the end of distillation, 50 mL (anhydrous/100%) toluene were added for preparation of a slurry (at approx. 100° C. and full speed agitation). Toluene has to be dried to a low water content before addition (e.g., with molsieve, AlOx or another appropriate method), or purchased in anhydrous form.

Cooling ramp (for laboratory trials) is maintained at −0.1 to 1 K/min to a minimum of 20° C. The cooling procedure must be adapted for considered equipment.

For washing the slurry is charged with pressure onto a filter. Residuals are transferred with 50 ml toluene (anhydrous) onto cake and filter cake is washed two times with 100 mL toluene (anhydrous), each. Afterwards, the filter cake was washed twice with 300 mL dry MTBE, each. The final product is dried with inert gas application and vacuum. Yield: 99.65% Purity Titration: 87.9 wt.-%.

Reaction of Benzyloxyethanol to Potassium-Benzyloxyethanolate with Potassium Tert-Butoxide

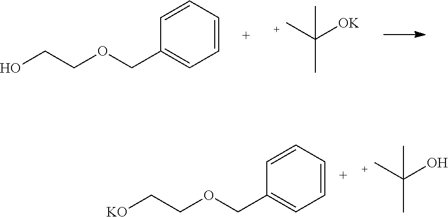

The desired reaction equipment is inertized and kept water-free prior conducting the reaction. 264.5 g benzyloxyethanol (99%; 1.7 mol; 1 Eq.) were charged into reactor, and 195.0 g potassium tert-butoxide (98%; 1.7 mol; 1 Eq.) where added, under inert (dry atmosphere). (Re)-inertization of reactor after addition of raw materials. The reaction was heated to 110° C. with constant stirring and nitrogen was started to be introduced into the mass. A 3-blade propeller stirring shaft with a total blade diameter of 75 mm was used. The nitrogen flow is maintained to approx. 100 L/h dry nitrogen for the whole duration of distillation. The tert-butyl alcohol was distilled off for approx. 120 h reaction time.

Full conversion is checked via NMR, with residual methanol content.

During distillation, stirring (rpm 100) becomes difficult because the viscosity becomes very high. The torque increases from 3-8 Ncm to 15-30 Ncm. The viscosity can be reduced by adding n-dodecane, (0.1-0.2 L) for a homogeneous reaction mixture, the torque decreases by the addition to 1-8 Ncm. n-dodecane is carried out at the reactor head as an aerosol during the reaction time and must be replenished in several portions. Combined amount of n-dodecane (100%; 0.75 L; 3.3 mol)

At the end of distillation, 50 mL (anhydrous/100%) toluene were added for preparation of a slurry (at approx. 100° C. and full speed agitation). Toluene has to be dried to a low water content before addition (e.g., with molsieve, AlOx or another appropriate method), or purchased in anhydrous form.

Cooling ramp (for laboratory trials) is maintained at −0.1 to 1 K/min to a minimum of 20° C. The cooling procedure must be adapted for considered equipment.

For washing the slurry is charged with pressure onto a filter. Residuals are transferred with 50 mL toluene (anhydrous) onto cake and filter cake is washed two times with 50 mL toluene (anhydrous), each. Afterwards, the filter cake was washed twice with 200 mL dry MTBE, each. The final product is dried with inert gas application and vacuum. Yield: 90.5% Purity Titration: 98.9 wt.-%.

### Reaction of Diethylenglycolmonobenzylether to Potassium-Diethylenglycolmonobenzylether

The desired reaction equipment is inertized and kept water-free prior conducting the reaction. 277.0 g diethylenglycolmonobenzylether (99%; 1.4 mol; 1 Eq.) were charged into reactor, and 98.0 g potassium methanolate (98%; 1.4 mol; 1 Eq.) where added, under inert (dry atmosphere). (Re)-inertization of reactor after addition of raw materials. The reaction was heated to 110° C. with constant stirring and nitrogen was started to be introduced into the mass. A 3-blade propeller stirring shaft with a total blade diameter of 75 mm was used. The nitrogen flow is maintained to approx. 100 L/h dry nitrogen for the whole duration of distillation. The methanol was distilled off for approx. 72 h reaction time.

Full conversion is checked via NMR, with residual methanol content.

During distillation, stirring (rpm 100) Reaction mass is a viscous liquid. The torque increases from 1-2 Ncm to 6-8 Ncm. The viscosity can be reduced by adding n-dodecane, (0.1-0.2 L) for a homogeneous reaction mixture, the torque decreases by the addition to 1-5 Ncm. n-dodecane is carried out at the reactor head as an aerosol during the reaction time and must be replenished in several portions. Combined amount of n-dodecane (100%; 0.75 L; 3.3 mol).

After the reaction, the product is present as a dark red highly viscous liquid. Yield: 74.8% Purity NMR: 91.3 wt.-%.

Reaction of 2-Tert-Butoxyethanole to Potassium-2-Tert-Butoxyethanolate with Potassium Tert-Butoxide+Variation of the Stirring Blade to Anchor Stirrers

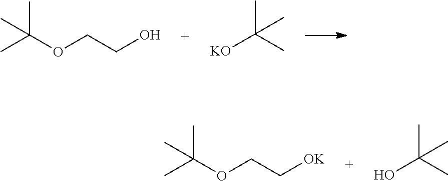

The desired reaction equipment is inertized and kept water-free prior conducting the reaction. 241.1 g 2-tert-butoxyethanole (98%; 2.0 mol; 1 Eq.) were charged into reactor, and 250.4 g potassium tert-butoxide (98%; 2.0 mol; 1 Eq.) where added, under inert (dry atmosphere). (Re)-inertization of reactor after addition of raw materials. The reaction was heated to 110° C. with constant stirring and nitrogen was started to be introduced into the mass. Anchor stirrers stirring shaft with a total blade diameter of 95 mm was used. The nitrogen flow is maintained to approx. 100 L/h dry nitrogen for the whole duration of distillation. The tert-Butyl alcohol was distilled off for approx. 26 h reaction time.

Full conversion is checked via NMR, with residual methanol content.

During distillation, stirring (rpm 100) becomes difficult because the viscosity becomes very high. The torque increases from 10-15 Ncm to 30-50 Ncm. The viscosity can be reduced by adding n-dodecane, (0.1-0.2 L) for a homogeneous reaction mixture, the torque decreases by the addition to 2-4 Ncm. n-dodecane is carried out at the reactor head as an aerosol during the reaction time and must be replenished in several portions. combined amount of n-dodecane (100%; 0.4 L; 1.8 mol)

At the end of distillation, 50 mL (anhydrous/100%) toluene were added for preparation of a slurry (at approx. 100° C. and full speed agitation). Toluene has to be dried to a low water content before addition (e.g., with molsieve, AlOx or another appropriate method), or purchased in anhydrous form. Enough dilution with toluene for transfer to filtration equipment is necessary, therefore the toluene content has to be adapted to existing filter equipment, also.

Cooling ramp (for laboratory trials) is maintained at −0.1 to 1 K/min to a minimum of 20° C. The cooling procedure must be adapted for considered equipment.

For washing the slurry is charged with pressure onto a filter. Residuals are transferred with 50 ml toluene (anhydrous) onto cake and filter cake is washed two times with 100 mL toluene (anhydrous), each. Afterwards, the filter cake was washed twice with 200 mL dry MTBE, each. The final product is dried with inert gas application and vacuum. Yield: 86.2% Purity Titration: 99.0 wt.-%.

Reaction of Triethylenglycol to Dipotassium-Triethylenglycolat with Potassium Tert-Butoxide+Variation of the Stirring Blade to Anchor Stirrers

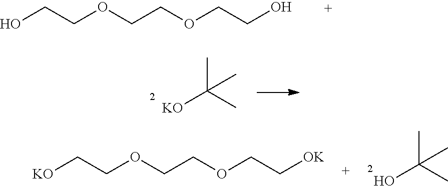

The desired reaction equipment is inertized and kept water-free prior conducting the reaction. 379.2 g triethylenglycol (99%; 2.5 mol; 1 Eq.) were charged into reactor, and 572.2 g potassium tert-butoxide (98%; 5.0 mol; 2 Eq.) where added, under inert (dry atmosphere). (Re)-inertization of reactor after addition of raw materials. The reaction was heated to 110°° C. with constant stirring and nitrogen was started to be introduced into the mass. Anchor stirrers stirring shaft with a total blade diameter of 95 mm was used. The nitrogen flow is maintained to approx. 100 L/h dry nitrogen for the whole duration of distillation. The tert-butyl alcohol was distilled off for approx. 77 h reaction time.

Full conversion is checked via NMR, with residual methanol content.

During distillation, stirring (rpm 100) becomes difficult because the viscosity becomes very high. The torque increases from 3-15 Ncm to 15-37 Ncm. The viscosity can be reduced by adding a mixture of n-dodecane and tetraglyme (v/v 50/50), (0.1-0.2 L) for a homogeneous reaction mixture, the torque decreases by the addition to 15-20 Ncm. The mixture is carried out at the reactor head as an aerosol during the reaction time and must be replenished in several portions. combined amount of n-dodecane (100%; 0.4L).

At the end of distillation, 50 mL (anhydrous/100%) toluene were added for preparation of a slurry (at approx. 100° C. and full speed agitation). Toluene has to be dried to a low water content, before addition (e.g., with molsieve, AlOx or another appropriate method), or purchased in anhydrous form Cooling ramp (for laboratory trials) is maintained at −0.1 to 1 K/min to a minimum of 20° C. The cooling procedure must be adapted for considered equipment.

For washing the slurry is charged with pressure onto a filter (filter area of 7-14 cm² resulting in a filter cake height of 16-8 cm). Residuals are transferred with 50 mL toluene (anhydrous) onto cake and filter cake is washed four times with 100 mL toluene (anhydrous), each. Afterwards, the filter cake was washed twice with 600 mL dry MTBE, each. The final product is dried with inert gas application and vacuum. Yield: 95.7% Purity Titration: 98.8 wt.-%.

Reaction of 2-Methoxethnaole to Potassium-2-Methoxyethanolat with Potassium Tert-Butoxide

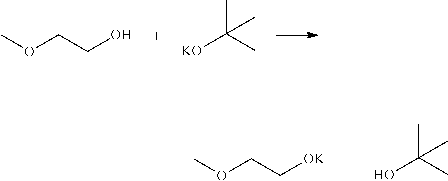

The desired reaction equipment is inertized and kept water-free prior conducting the reaction. 305.0 g triethylenglycol (99%; 4.0 mol; 1 Eq.) were charged into reactor, and 458.0 g potassium tert-butoxide (98%; 4.0 mol; 1 Eq.) where added, under inert (dry atmosphere). (Re)-inertization of reactor after addition of raw materials. The reaction was heated to 120° C. with constant stirring and nitrogen was started to be introduced into the mass. A 3-blade propeller stirring shaft with a total blade diameter of 95 mm was used. The nitrogen flow is maintained to approx. 100 L/h dry nitrogen for the whole duration of distillation. The tert-Butyl alcohol was distilled off for approx. 50 h reaction time.

Full conversion is checked via NMR, with residual methanol content.

During distillation, stirring (rpm 100) becomes difficult because the viscosity becomes very high. The torque increases from 3-5 Ncm to 15-20 Ncm. The viscosity can be reduced by adding of n-dodecane, (0.1-0.2 L) for a homogeneous reaction mixture, the torque decreases by the addition to 2-5 Ncm. The mixture is carried out at the reactor head as an aerosol during the reaction time and must be replenished in several portions. combined amount of n-dodecane (100%; 0.5L).

At the end of distillation, 50 mL (anhydrous/100%) toluene were added for preparation of a slurry (at approx. 100° C. and full speed agitation). Toluene has to be dried to a low water content before addition (e.g., with molsieve, AlOx or another appropriate method), or purchased in anhydrous form.

Cooling ramp (for laboratory trials) is maintained at −0.1 to 1 K/min to a minimum of 20° C. The cooling procedure must be adapted for considered equipment.

For washing the slurry is charged with pressure onto a filter. Residuals are transferred with 50 mL toluene (anhydrous) onto cake and filter cake is washed two times with 50 mL toluene (anhydrous), each. Afterwards, the filter cake was washed twice with 200 mL dry MTBE, each. The final product is dried with inert gas application and vacuum. Yield: 93.7% Purity Titration: 97.1 wt.-%.

Reaction of Pentaerythritol to Tetrapotassium-Pentaerythritolate with Potassium-Methoxide

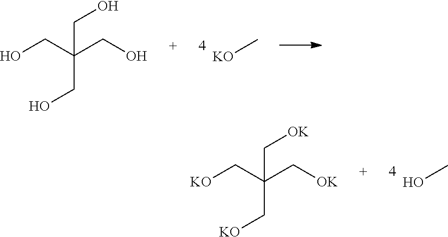

The desired reaction equipment is inertized and kept water-free prior conducting the reaction. 277.8 g pentaerythritol (99%; 2.0 mol; 1 Eq.) were charged into reactor, and 567.0 g potassium tert-butoxide (98%; 8.1 mol; 4 Eq.) where added, under inert (dry atmosphere). (Re)-inertization of reactor after addition of raw materials. The reaction was heated to 145° C. with constant stirring and nitrogen was started to be introduced into the mass. A 3-blade propeller stirring shaft with a total blade diameter of 95 mm was used. The nitrogen flow is maintained to approx. 100 L/h dry nitrogen for the whole duration of distillation. The methanol was distilled off for approx. 390 h reaction time.

Full conversion is checked via NMR, with residual methanol content.

During distillation, stirring (rpm 100) becomes difficult because the viscosity becomes very high. The torque increases from 3-5 Ncm to 15-20 Ncm. The viscosity can be reduced by adding a mixture of n-dodecane and Diglyme (v/v 50/50), (0.1-0.3 L) for a homogeneous reaction mixture, the torque decreases by the addition to 2-8 Ncm. The mixture is carried out at the reactor head as an aerosol during the reaction time and must be replenished in several portions. Combined amount of n-dodecane (100%; 1.6L).

At the end of distillation, 50 mL (anhydrous/100%) toluene were added for preparation of a slurry (at approx. 100° C. and full speed agitation). Toluene has to be dried to a low water content, before addition (e.g., with molsieve, AlOx or another appropriate method), or purchased in anhydrous form.

Cooling ramp (for laboratory trials) is maintained at-0.1 to 1 K/min to a minimum of 20° C. The cooling procedure must be adapted for considered equipment.

For washing the slurry is charged with pressure onto a filter. Residuals are transferred with 50 ml toluene (anhydrous) onto cake and filter cake is washed three times with 50 mL toluene (anhydrous), each. Afterwards, the filter cake was washed twice with 200 mL dry MTBE, each. The final product is dried with inert gas application and vacuum. Yield: 55.2% Purity Titration: 69.4 wt.-%.

For washing the slurry is charged with pressure onto a filter and the filter cake is washed two times with non-polar solvent. The final product is dried with inert gas under application and vacuum.

### Reaction of Benzyloxyethanol to Benzyloxyethanolate in Presence of N-Dodecane

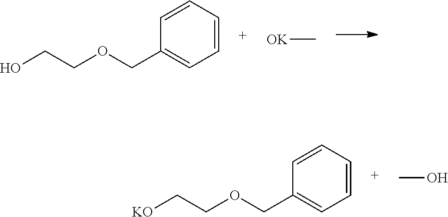

The desired reaction equipment is inertized and kept water-free prior conducting the reaction.

627.7 g benzyloxyethanol (98%; 4 mol; 1 Eq.) were charged into reactor, and 166.8 g potassium methanolate (98.95%; 4; 1 Eq.) where added, under inert (dry atmosphere). (Re)-inertization of reactor after addition of raw materials. The reaction was heated to 110° C., and it was started to introduce nitrogen into the melt. The nitrogen flow is maintained to approx. 100 L/h dry nitrogen for the whole duration of distillation. The methanol was distilled off for approx. 44h reaction time.

Full conversion is checked via NMR, with residual methanol content.

During the distillation stirring is getting difficult, as viscosity is getting very high (indicated by increase of stirrer torque). The viscosity can be reduced by addition of n-dodecan, (100%; 0.16 kg; 0.215 L) for a resulting in a homogeneous reaction mixture. n-dodecane is carried out at reactor head as an aerosol, during reaction period, and has to be replenished in several portions. summarized amount of n-dodecane (100%; 250 ml; 1.1 mol; 1.1 Eq)

At the end of distillation, 250 ml (anhydrous/100%) toluene were added for preparation of a slurry (at approx. 100° C. and full speed agitation). Toluene has to be dried to a low water content before addition (e.g., with molsieve, AlOx or another appropriate method), or purchased in anhydrous form. Enough dilution with toluene for transfer to filtration equipment is necessary, therefore the toluene content has to be adapted to existing filter equipment, also.

Cooling ramp (for laboratory trials) is maintained at −0.1 to 1 K/min to a minimum of 20° C. The cooling procedure must be adapted for considered equipment.

For washing the slurry is charged with pressure onto a filter. Residuals are transferred with 125 mL toluene (anhydrous) onto cake and filter cake is washed two times with 125 mL toluene (anhydrous), each. Afterwards, the filter cake was washed twice with 200 ml dry MTBE, each. The final product is dried with inert gas application and vacuum. Bulk density (dry product) is approx. 0.94 kg/l.

Reaction of Benzyloxyethanol to Potassium Benzyloxyethanolate with Potassium Tert-Butoxide in a Continuous Distillation

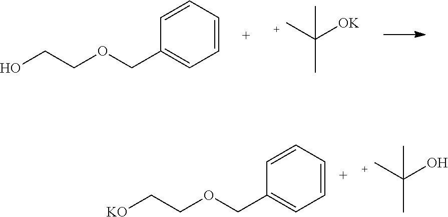

The desired reaction equipment is inertized and kept water-free prior to conducting the reaction. 232.7 g benzyloxyethanol (99%; 1.5 mol; 1 Eq.) were charged into reactor, and 191.6 g potassium tert-butoxide (98%; 1.5 mol; 1 Eq.) where added under inert and dry atmosphere, followed by inertization of reactor. The reaction was heated to 110° C. with constant stirring and nitrogen was started to be introduced into the mass. An anchor stirrer with a total blade diameter of 90 mm was used. A capillary was used to constantly introduce toluene into the reaction mass (1 mL/minute). The nitrogen flow was maintained at approx. 100 L/h dry nitrogen for the whole duration of distillation. tert-Butyl alcohol and toluene were distilled off for approx. 5.5 h reaction time, during which a thicker suspension is obtained. The reaction is terminated by lowering the temperature to 20° C., the mass is diluted after 6 hours and can be homogeneously drained from the reaction vessel.

The cooling ramp is maintained at −0.1 to 1 K/min to a minimum of 20° C. The cooling procedure must be adapted for considered equipment.

The slurry was charged with pressure onto a filter. Residuals were transferred with 50 mL toluene (anhydrous) and the filter cake was washed two times with 50 mL toluene (anhydrous) each. Afterwards, the filter cake was washed twice with 200 mL dry MTBE each. The final product was dried under inert conditions and under the application of vacuum. Yield: 90.1% Purity Titration: 99.1 wt.-%.

1. A method of producing an alkali metal alkoxide, the method comprising:
i) providing an organic compound having at least one —OH group and having a molecular weight of 75 to 750 g/mol in a reaction vessel in an inert atmosphere;
ii) adding a C₁₋₆ alkali metal alcoholate to obtain a reaction mixture;
iii) heating the reaction mixture of step ii) under stirring to 25 to 180° C. to obtain the alkali metal alkoxide and a C₁₋₆ alcohol; and
iv) distilling off the obtained C₁₋₆ alcohol; wherein while distilling, at least one non-polar solvent having a boiling point higher than the obtained alcohol, is continuously or batchwise added to the mixture.

2. The method of claim 1, wherein the organic compound having at least one —OH group is selected from the group consisting of substituted or unsubstituted aliphatic or aromatic hydrocarbons, silyl ethers, polyols, ethers, and polyethers or mixtures thereof.

3. The method of any of the preceding claims claim 1, wherein the C₁₋₆ alkali metal alcoholate is selected from the group consisting of potassium methanolate, potassium ethanolate, potassium 2-methylpropan-2-olate, potassium propan-2-olate, potassium propan-1-olate, potassium butan-2-olate, and potassium tert-butoxide.

4. The method of claim 1, wherein the at least one solvent is non-polar and aprotic.

5. The method of claim 1, wherein the heating in iii) comprises heating in a reactor, and wherein the reactor comprises a rotating element for the stirring.

6. The method according to claim 1, wherein the at least one non-polar solvent is present in an amount of 1 ml/mmol to 20 ml/mmol to the organic compound having at least one —OH group and having a molecular weight of 75 to 750 g/mol.

7. The method according to claim 1, wherein a reaction time for iii) is at least 3 hours.

8. The method according to claim 1, wherein the at least one non-polar solvent is washed out after the reaction with a volatile non-polar solvent.

9. The method according to claim 1, wherein the organic compound having at least one —OH group and having a molecular weight of 75 to 750 g/mol and the C₁₋₆ alkali metal alcoholate are present in a molar ratio of 2:1 to 1:1.

10. The method of claim 4, wherein the at least one solvent is selected from the group consisting of aromatic hydrocarbons, aliphatic hydrocarbons, and ether solvents.

11. The method of claim 5, wherein the at least one non-polar solvent is added, when the moment of force M of the rotating element has increased by a factor of at least 1.5 and the moment of force M is reduced after addition of the at least one non-polar solvent by a factor of at least 1.5.
